(19) United States
(12) Patent Application Publication
(10) Pub. No.: US 2022/0148693 A1
(43) Pub. Date: May 12, 2022

(54) PATENT-CENTRIC HEALTH CARE SYSTEM

(71) Applicant: Samuel Herzog, Montreal (CA)

(72) Inventor: Samuel Herzog, Montreal (CA)

(21) Appl. No.: 17/389,348

(22) PCT Filed: Jan. 30, 2020
(86) PCT No.: PCT/IB2020/050745

§ 371 Date: Jul. 30, 2021

Related U.S. Application Data
(60) Provisional application No. 62/799,056, filed on Jan. 31, 2019.

(51) Int. Cl. G16H 10/60; G16H 80/00; G16H 40/20

(52) U.S. Cl. CPC G16H 10/60; G16H 40/20; G16H 80/00

(57) ABSTRACT

In the field of end-to-end healthcare management there exists an issue with system inefficiencies as well as the lack of fluidity with which a patient's medical history records can be shared in order to provide optimum, efficient and effective healthcare delivery. A system for patient-centric health care is provided, comprising: a plurality of client computing devices configured to store medical records, and to provide interactive patient medical management systems for each of a plurality of patients and a centralized host server which is configured to store medical records and to provide communication interfaces with interactive medical management systems for each of a plurality of patients, hospitals, clinics, medical test centers, physicians, pharmacies, insurance companies and patients.

### FIELD OF THE INVENTION

The present invention relates to an end-to-end health care system.

### BACKGROUND OF THE INVENTION

Health care delivery today takes a top-down approach, designed around care providers, such as physicians, and care provider facilities, such as hospitals and clinics. Patients are ostensibly in control of their own healthcare. Thus someone may schedule an appointment with his family physician, show-up at an emergency room or a walk-in clinic, or visit a specialist referred by a general practitioner or an emergency room doctor. At all stages the patient is entitled to make his own decisions about his health care; however, the information needed in order to make these decisions is most often held by providers, thus limiting the patient's ability to make optimal decisions. Patients are also limited in their ability to make choices regarding the manner in which their health care is delivered and by whom. It is difficult, for example, to make a fully informed decision when selecting a family physician or specialist. It is also difficult to make informed decisions about different treatment or care options.

Furthermore, inefficiencies in the current system mean that patients are not being provided with timely access to health care. There is little real-time information available regarding healthcare demand vs. healthcare resources. Hospitals and emergency rooms regularly have long waits, but there is no ability to coordinate between facilities for optimization of wait times. Often the wait time for medical testing, such as blood tests and imaging, is long, while devices or facilities for performing the medical tests are under-utilized. Test results may not be timely reviewed by the prescribing physician, yet other physicians may be available who can perform the review.

In addition, patients typically are not informed as to whether their medical tests have been completed or have been reviewed by the appropriate medical professional, unless and until their physician contacts them. This drawback leaves patients in a state of uncertainty regarding their health status and health care decisions that need to be made. This drawback has adverse effects on health care outcomes.

A patient's medical information is gathered by each health care provider or each health care provider's organization, for example a clinic or hospital, and the information is stored in the electronic health records system of each organization separately. Typically, the information is not shared in any cooperative manner, and thus the patient's health records are dispersed across multiple sources and are not easily accessible if the patient or the patient's health care provider wants a comprehensive view of the patient's medical history. A physician who does not have access to the patient's complete medical history wastes time by having to obtain further information from the patient or from other sources, and may make decisions without the benefit of full knowledge. This drawback too has adverse effects on healthcare outcomes.

Society as a whole, including governments, does not have real-time or comprehensive information about the health care system and health care delivery. Information is gathered and made available with large expenditures of time and money, and by the time any valuable insights are gleaned from the data the realities may have already changed. This drawback impedes the progress of health care research and also leaves unexplored many potential avenues for improving and optimizing the healthcare delivery ecosystem.

There is therefore a need for an improved system for the delivery of health care.

### SUMMARY

A key to the improved system disclosed herein is a shift of control from the current diffuse, multi-point professional-oriented system to a re-conceived system in which the locus of information flow and the control over access to health care resources is centered on the patient. Under embodiments of the present invention, a profile is created for each patient within a patient-centric health care (PCH) system, in which all of the patient's medical history is collected. Health care professionals are granted access to the profile, in whole or in part, as required. Patients maintain ultimate management over their health records and access, but such management is done in conjunction with the relevant professionals. Within the PCH system, the relevant professional may facilitate access of the patient profile to other professionals and resources.

A health care professional refers a patient to other resources as required, such as referral to a specialist, ordering a test, and prescribing medication. Under the PCH system, the patient with a referral for a particular type of care or service is presented with a list of options based upon up-to-date information on the availability of the relevant health care service by geographic area, expertise, experience, languages spoken and/or other parameters. The selection process is guided by the referring professional, whose referral opens relevant information to the patient, but the patient has the ability to select the resource of his or her choice through the PCH system.

The availability of data on health care resources by geographic area, wait times and other relevant indicators also gives professionals better insight into resource availability and leads to more efficient use of existing resources. Furthermore, society at large will have more knowledge of medical treatment options.

The various aspects of the proposed PCH system are described below.

There is thus provided in accordance with an embodiment of the present invention a system for patient-centric health care, including a plurality of client computing devices configured to store medical records, and to provide interactive patient medical management systems (MMSs) for each of a plurality of patients, and a centralized host server configured to provide communication interfaces with interactive MMSs for each of a plurality of hospitals, clinics, medical test centers, physicians, pharmacies, insurance companies and patients, wherein the client computers and the centralized host server include processors and instructions that enable each patient MMS to perform the following processes: (a) share patient medical records with hospitals, clinics, medical test centers, physicians, pharmacies, insurance companies or emergency responders, (b) schedule appointment with a physician, (c) schedule an appointment with a specialist based on a referral from a physician, (d) schedule a medical test based on a referral from a physician, (e) transmit a prescription prescribed by a physician to a pharmacy for fulfillment, and (f) generate an insurance claim.

There is additionally provided in accordance with an embodiment of the present invention a system for patient-centric health care that provides each of a plurality of patients with access to his medical records, including physician reports, hospital reports, test results, treatments, medications, prescriptions and insurance claims, and enables each patient to share access to at least a portion of his medical records with hospitals, clinics, medical test centers, physicians, pharmacies, insurance companies and emergency responders.

### BRIEF DESCRIPTION OF THE DRAWINGS

The present invention will be more fully understood and appreciated from the following detailed description, taken in conjunction with the drawings in which:

FIG. 1 is a simplified top-level architecture diagram of a patient-centric health care system system, in accordance with an embodiment of the present invention;

FIG. 2 is a simplified series of screenshots showing how a patient permits specific physicians, clinics, hospitals and laboratories and other organizations to access the patient's medical records, in accordance with an embodiment of the present invention;

FIG. 3 is a simplified screenshot of a home screen of a patient profile, in accordance with an embodiment of the present invention;

FIG. 4 is a simplified screenshot of a physician's view of a patient profile, in accordance with an embodiment of the present invention;

FIG. 5 is a simplified diagram of an exemplary patient-centric healthcare system, in accordance with an embodiment of the present invention;

FIG. 6 is a simplified series of screenshots showing how a patient searches for and books an appointment with a physician, in accordance with an embodiment of the present invention;

FIG. 7 is a simplified series of screenshots showing how a patient views available referrals, in accordance with an embodiment of the present invention;

FIG. 8 is a simplified series of screenshots showing how a patient has a prescription filled, in accordance with an embodiment of the present invention;

FIG. 9 is a simplified series of screenshots showing how a medical test is requested and how the test results are provided to the patient, in accordance with an embodiment of the present invention;

FIG. 10 is a simplified series of screenshots showing how a physician books a clinic for a specific medical practice based on patient needs and available facilities, in accordance with an embodiment of the present invention; and

FIG. 11 is a simplified screenshot showing real-time emergency room information for a given geographical location, in accordance with an embodiment of the present invention.

### DETAILED DESCRIPTION

I. System and Specification Dichotomy

The patient-specific health care (PCH) system includes both a set of specifications and an implementation per those specifications. By adhering to the specifications, multiple entities may develop and operate different parts of the PCH system, while still allowing the PCH system as a whole to operate as described herein.

The PCH system specifications include an authentication specification. The authentication specification specifies how consumer-facing components of the system allow consumers, i.e., patients, to authenticate themselves via the PCH system. The authentication specification further specifies how healthcare providers, such as physicians, clinics, hospitals, and pharmacies, authenticate themselves via the PCH system.

The authentication specification includes a multi-tiered authorization mechanism whereby different authenticating identities are granted different levels of authorization to access PCH system resources. Thus, for example, a patient has full authority over his own health data, including the right to delegate access to that data to other individuals or healthcare providers, while a physician has access to the health data of any patient under his current care but, under typical circumstances, does not have authorization to delegate access to the data to other individuals or organizations. Similarly, a clinic has access to data relating to the clinic's physicians and patients under the care of those physicians.

The authentication specification includes a mechanism for federated authentication, which allows multiple third-party implementors of different aspects of the system to provide authentication features to the users of their respective PCH system aspects.

The PCH system specifications include a health record format specification. The health record format specification specifies in what electronic formats patient health records are stored in the system, and are transferred between different components of the system, including components that were built by and are operated by multiple entities. The health record format specifications rely on existing and future open specifications. However, the health record format specification, in some cases, defines proprietary record format specifications.

The PCH system specifications include a health record monetization specification. The health record monetization specification specifies how maintenance of health records and transfer of health records from one system component to another are monetized by the maintainers and transferrers of the records. Operation of the PCH system does not require that any activities be monetized; however, the system does allow for monetization.

The PCH system specifications include a health record transfer specification. The health record transfer specification specifies protocols or other technical mechanisms that must be followed by operators of different components of the PCH system, in order to transfer health records from one component to another.

The PCH system specifications include a communication specification. The communication specification specifies how different components of the system communicate with one another, including components that were built or are operated by different entities. The PCH system is designed as a loosely-coupled service-oriented system, whereby any component can communicate with, and request services of, multiple other components of the system. Communication between different components is via web services or similar mechanism, which allows software products to communicate with each other in a platform-agnostic, geographically-agnostic manner.

The PCH system specifications include a certification specification. The certification specification specifies a set of implementation requirements, functional requirements, performance requirements, user interface requirements, data handling requirements, security requirements, and other requirements that third party implementors must adhere to in order to build or operate different components of the PCH system.

The certification specification specifies a set of tests to be performed on components of the PCH system, and a set of conditions that must be met on the results of the tests performed, in order to ascertain whether a component is certified for operation as part of the PCH system.

II. Architecture

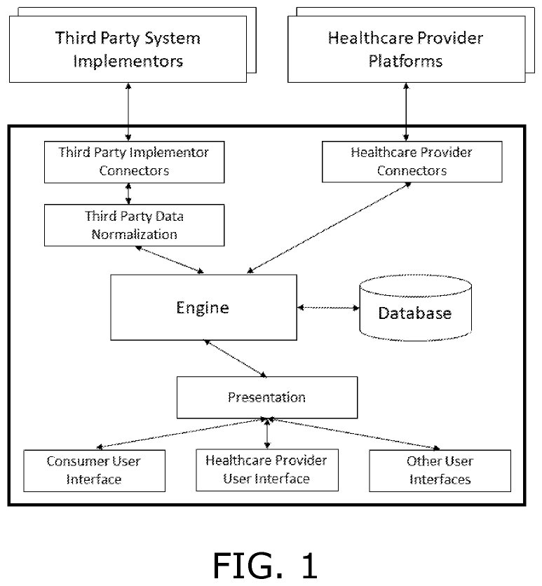

Reference is made to FIG. 1, which is a top-level architecture diagram of a PCH system, in accordance with an embodiment of the present invention. All components inside the heavy black box of FIG. 1 are parts of the PCH system itself, while those outside the box are systems developed by third parties.

At the top right of FIG. 1 are shown various health care providers and related platforms, including inter alia clinics, hospitals, insurance companies and emergency response systems, that integrate with the PCH system. The providers and platforms integrate via the connectors as shown.

At the top left of FIG. 1 are shown third party implementors of parts of the PCH system. Thus in some circumstances a third party maintains some segment of a patient's health records and makes the records available to the patient through the PCH system. These implementors integrate with the PCH system through their own set of connectors, as shown. Information maintained by such third parties is normalized by the PCH system and presented to patients and health care providers in a uniform manner. Thus it is transparent to a particular user or healthcare provider where exactly a given piece of information resides.

In the center of FIG. 1 are shown the engine of the PCH system and the PCH system's own data store.

In the lower portion of FIG. 1 are shown the presentation layer and user interfaces to the various types of PCH system users. These users include inter alia patients, physicians, clinics, hospitals, insurance companies and emergency response organizations.

III. Patent-Driven Health Record Management

A given patient's medical and health record is associated with his own patient profile. The patient has complete control over what information goes in to the health record, and how the information is accessed by others.

The health record aggregates information from a wide variety of sources, including a personal physician, specialists, clinics, diagnostic labs, hospitals and pharmacies. In addition, the health record aggregates information from a patient's own devices. These devices include fitness trackers, sleep trackers, home diagnostic devices such as blood pressure monitors, and other consumer devices. The patient manually adds to the health record any information that is pertinent but is not aggregated automatically by the PCH system. This information includes health events occurring overseas, i.e., in areas not covered by the PCH system, and self-reported symptoms.

When a patient requests services of physicians, clinics, hospitals and laboratory services, all the events, tests and associated data are automatically added to the health record associated with the patient's profile.

The patient may choose to block certain information from being entered into the health record. Such blocked information may include information the patient considers to be of a particularly sensitive or personal nature.

In situations where it is medically pertinent, the PCH system allows physicians or other health care providers to keep certain information out of the patient's health record. Such information includes inter alia notes on patient behavior such as aggression towards medical staff, and non-compliance with treatment plans.

The patient controls how information in the profile is shared with others. Information may be shared with health care providers and health care organizations, and may also be shared with family members, patient advocates, or other designees. In addition, information in the profile may be shared with insurance companies.

The patient also controls downstream dissemination of information by health care providers to other health care providers. For example, a patient may opt to share all information with his family physician but with no other providers. He may opt to share with his physician, but in case of an emergency to share with anyone providing emergency care. He may delegate authority to his physician to share his information with other health care providers as the physician sees fit, or may limit the ways in which, or the providers to which, the physician may share his information. Alternatively, he may choose to share all information with all health care professionals involved in his care. He may also opt to share all or certain information with a parent, child, friend, or advocate.

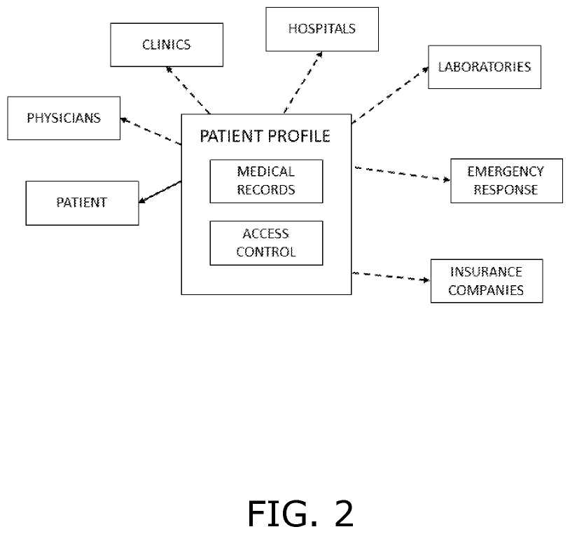

Reference is made to FIG. 2, which a simplified series of screenshots showing how a patient permits specific physicians, clinics, hospitals and laboratories and other organizations to access the patient's medical records, in accordance with an embodiment of the present invention. The patient specifies which records are visible to each health care provider or organization and under what circumstances.

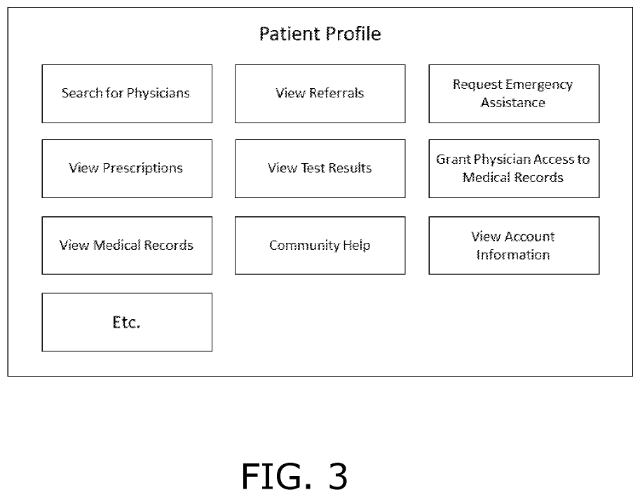

Reference is made to FIG. 3, which is a simplified screenshot of a home screen of a patient profile, in accordance with an embodiment of the present invention. When the patient logs in to the PCH system, the patient is presented with his or her profile and various options. FIG. 3 illustrates some specific and non-limiting examples of options that are available.

A physician, having access granted by the patient, views the patient's complete medical record, which allows the physician to assess the patient more accurately and in less time, as there is no need to gather information from the patient.

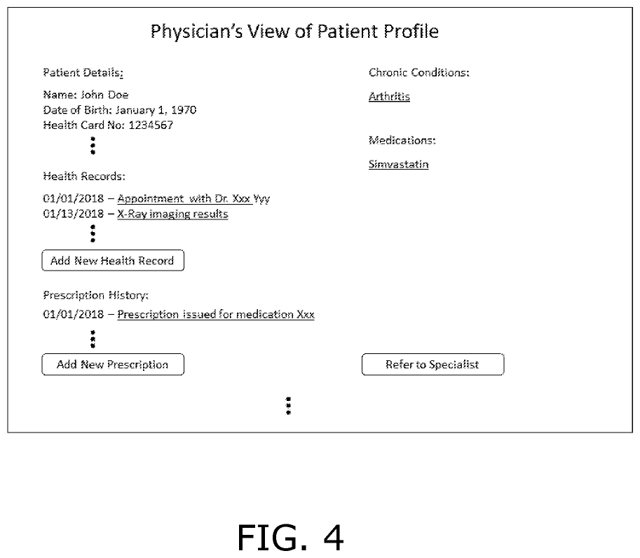

Reference is made to FIG. 4, which is a simplified screenshot of a physician's view of a patient profile, in accordance with an embodiment of the present invention. The physician is prompted by the PCH system to view the profile in response to an event such as an upcoming appointment. Alternatively, the physician opens the patient's profile manually. Once the physician has accessed the profile, the physician views the patient's medical records, communicates new requests or events, views the patient's prescriptions, adds a new prescription, and/or refers the patient to a specialist.

If the patient visits a new health care provider outside of his normally accessed care network, namely, his family physician and local pharmacy, the patient may grant to the new health care provider, or authorize the referring physician to grant to the new health care provider, access to the patient's profile. The new health care provider will then have access, in whole or in part, to the patient's medical history. Any new medical information generated by the new provider is added to the patient's records, thus allowing the patient to have worldwide control of his medical records.

IV. Centralized Records

A consequence of having patient-driven medical records is that the storage of the medical records can be centralized.

In certain situations or countries it may be desirous, or even required, that patient health records be maintained in a centralized manner. The system allows for and facilitates centralized record keeping.

In other situations, for example where a free-market approach to health care is taken, the PCH system facilitates the distributed storage and maintenance of medical records. Multiple providers maintain different segments of a patient's health data but through the PCH system the data is presented to the patient as a complete health record. Thus, from the patient's perspective the information is centralized and fully under his control, while the actual data may in fact be distributed.

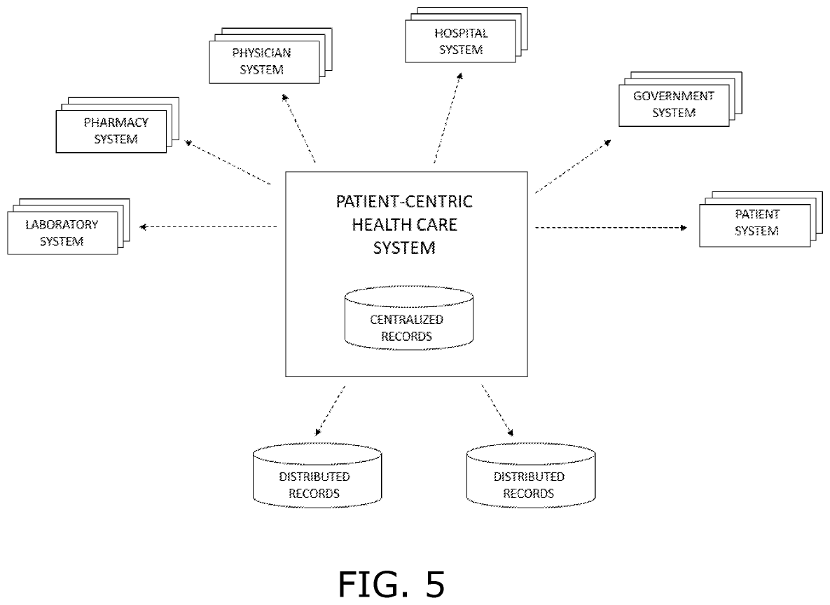

Reference is made to FIG. 5, which is a simplified diagram of an exemplary PCH system, in accordance with an embodiment of the present invention. The PCH system captures all health care records and events of patients from various sources, such as from physicians, hospitals, pharmacies, laboratories, governments and patients. The PCH system stores captured health care records in a centralized data store and in a plurality of distributed data stores.

In addition to capturing and maintaining healthcare records of patients, the PCH system also maintains lists of health care related resources, including inter alia lists of medical facilities such as clinics and hospitals, and lists of health care providers such as physicians and nurses. In addition the PCH system maintains an inventory of medical supplies and equipment available at medical facilities, such as diagnostic supplies, medications, and various types of medical devices.

V. Appointment Options

The patient uses the PCH system to search for physicians that are accepting new patients or for specialists of a given type. The patient is able to search based on geographical location, based on availability, e.g., a doctor is available on a certain day, based on languages spoken, or based on a range of other criteria. The patient selects a given physician and sees that physician's availability, and the rating of the physician from other patients. After selecting a physician, the patient uses the PCH system to make an appointment.

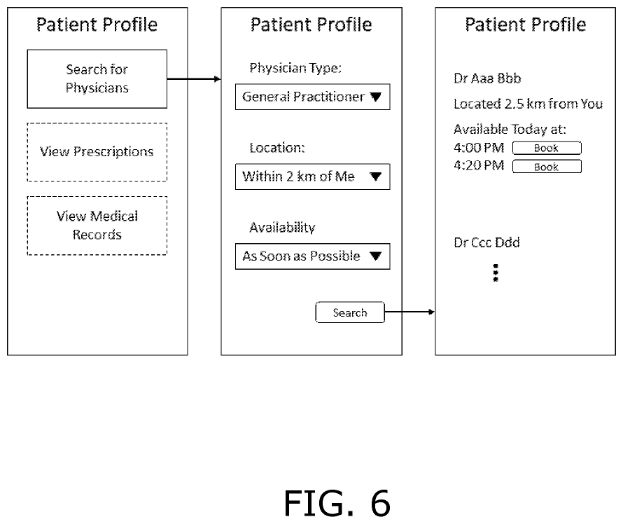

Reference is made to FIG. 6, which is a simplified series of screenshots showing how a patient searches for and books an appointment with a physician, in accordance with an embodiment of the present invention. The patient selects “Search for Physicians” on the home screen of the patient's profile. The patient then searches for physicians of a given type, location and availability. The patient then views the available physicians and books an appointment. The patient's medical records are automatically made available to the booked physician.

The PCH system allows for last-minute cancellations of an appointment to be shown as an available appointment, allowing for the appointment slot to be potentially re-booked by another patient.

VI. Referral Process

A physician refers a patient to a given type of specialist, e.g., a dermatologist on the PCH system. The referral is added to the patient's profile on the PCH system. The PCH system then generates options for how the patient proceeds to book an appointment with a specialist. The patient may search for all specialists, e.g., all dermatologists, available for that referral. The search may be done for all specialists that are within a certain geographical location, by availability, and/or by language or other criteria. The patient selects a given specialist to see the appointment openings of that specialist, and books an appointment using the PCH system, as described in the “Appointment Options” section hereinabove.

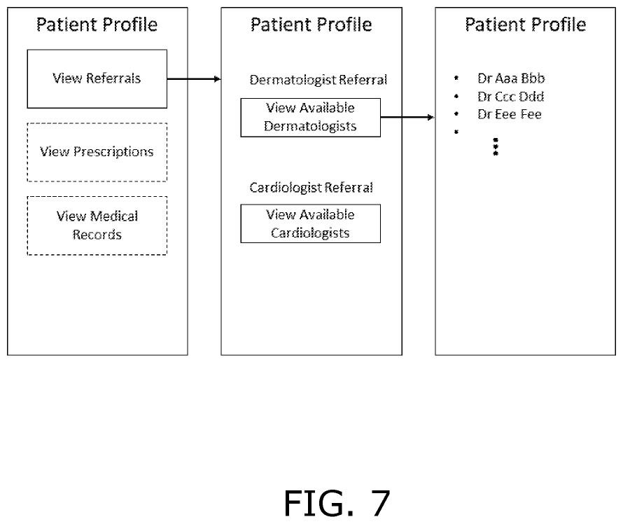

Reference is made to FIG. 7, which is a simplified series of screenshots showing how a patient views available referrals, in accordance with an embodiment of the present invention. By selecting “View Referrals” on the home screen of the patient's profile, the patient sees that he has a referral to a dermatologist and views the available dermatologists.

The patient is able to book an appointment with a physician for the purpose of getting a referral. The patient is able to select the type of specialist that the patient would like to see, and to book an appointment with an available physician who can provide the referral. For example, a telemedicine appointment, e.g., video chat, may be made with a currently available physician, and the patient is able to get a referral to a specialist without having to travel to his family physician's clinic.

After a patient has booked an appointment with a specialist through the referral process, the referring physician selects which medical records are to be provided to the specialist. Alternatively, the patient may grant the specialist access to all of his medical records. In this manner relevant medical records of the patient are automatically accessible to the specialist, without the specialist needing to request them or take any action.

VII. Prescription Delivery

A physician may prescribe a prescription drug to a patient on the PCH system. The prescription is added to the patient's profile. The system generates options for how the patient can have the prescription filled and the patient has the PCH system send the prescription to a given pharmacy. For example, the patient may select a pharmacy based on geographical location, or may have the prescription automatically directed to a preferred pharmacy. The system automatically reviews the patient's medical records to identify any drug interactions or allergies with the prescribed medication. The system alerts the physician or the pharmacist, and the prescription may be modified as necessary. Once filled, the prescription is picked up by the patient or shipped to the patient.

The patient grants the pharmacy access to the patient's prescription history and to any other relevant medical records. The pharmacist is then able to assess for allergies, drug interactions and the like. If the pharmacist discovers any issues that were not identified by the PCH system automatically, the pharmacist notifies the prescribing physician using the PCH system, so that the prescribing physician can update the prescription to a more suitable alternative. The PCH system provides the patient with a notification when the prescription is ready for pick-up, and the patient can pick up his medication at the pharmacy.

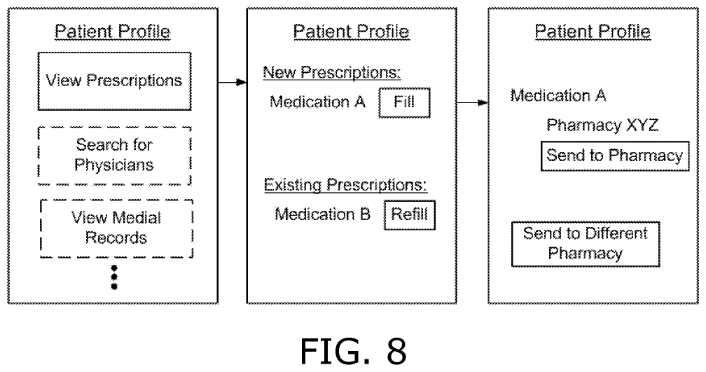

Reference is made to FIG. 8, which is a simplified series of screenshots showing how a patient has a prescription filled, in accordance with an embodiment of the present invention. The patient selects “View Prescriptions” from the home screen of the patient's profile, views any new prescriptions, and selects the prescription to be filled. The PCH system confirms that the patient would like to have the prescription filled by the patient's default pharmacy, or the patient selects a different pharmacy to fill the prescription.

The PCH system is used by the patient to request a prescription refill from his doctor. If an appointment with a physician is required for the prescription refill, the PCH system sets up an appointment with the patient and the physician, either a physical appointment or a telemedicine appointment, to have the prescription refilled. If the prescribing physician is not available, an appointment is made with a currently available physician, who is granted access to the patient's medical history. The selected physician then refills the prescription, and the PCH system sends the prescription to the pharmacy.

If an appointment is not required, the patient's refill request is sent by the PCH system directly to the physician, who then refills the prescription. The PCT systems sends the refill prescription to the pharmacy.

VIII. Laboratory Testing

Using the PCH system, a physician requests that a patient undergo a specific medical test, e.g., a blood test, a urine test, or imaging. The requested medical test is added to the patient's profile on the PCH system. The PCH system generates options for how the patient can have the medical test performed. For example, the patient searches for a nearby laboratory that is able to perform the given medical test, and books an appointment using the PCH system.

Once a test is performed, the results are provided by the laboratory to the PCH system, and the patient is notified that the test results are complete. If the test results do not require interpretation by a medical professional, the test results are made available to the patient by the PCH system when the test results are ready. If the test results require interpretation by a medical professional, the test results are sent by the PCH system to the prescribing physician. If the prescribing physician is not available, the PCH system sends the results to another physician who is currently available to perform a review. Alternatively, the PCH system allows some time for the prescribing physician to become available to review the results, and if the physician does not become available within a predetermined time frame the PCH system instead provides the results to an alternate physician who is currently available.

When a physician review is pending, the PCH system notifies the patient that the results are pending review. After the review is completed, the test results along with any interpretation or physician notes are added to the patient's profile. The patient may grant access to the test results to another care provider for a second assessment and opinion.

A patient may request an appointment with a physician for the specific purpose of requesting a particular medical test. In other words, when booking the appointment through the PCH system, the patient indicates that he would like the physician to prescribe the specific medical test. For example, a telemedicine appointment, e.g., video chat, may be made with a currently available physician and the patient is able to get a referral for the medical test without having to travel to a clinic for the referral.

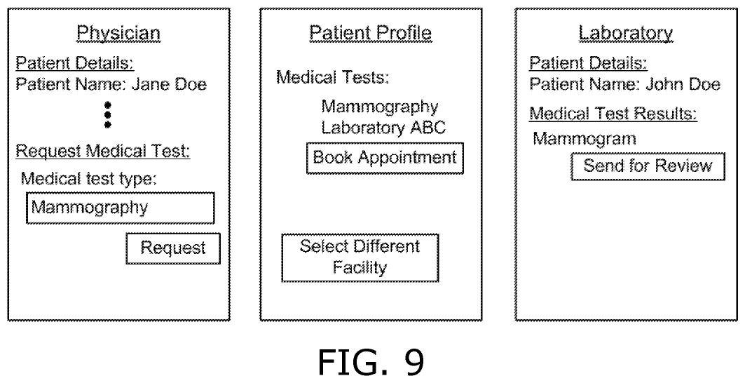

Reference is made to FIG. 9, which is a simplified series of screenshots showing how a medical test is requested and how the test results are provided to the patient, in accordance with an embodiment of the present invention. As shown in the leftmost screenshot, the physician prescribes a specific medical test for a patient. The prescribed medical test is then made available in the patient's profile, as shown in the middle screenshot. The patient then selects the facility at which the medical test is to be performed. As shown in the rightmost screenshot, the testing facility connects to the PCH system and sends the test results to the physician for review via the PCH system. The patient follows the process step by step using the PCH system.

IX. Health and Treatment Data Analysis

The PCH system performs analysis of health records using various statistical and machine learning algorithms in order to find optimal treatment options and improve healthcare outcomes.

Pursuant to the regulatory environment in any given region, the PCH system is granted access to anonymized patient data and health record data for all patients using the PCH system. When not granted access automatically, the PCH system is granted access explicitly on an opt-in basis by patients. The PCH system may offer incentives to patients to make their anonymized data available for analysis.

The PCH system itself performs analyses on health care data. Alternatively, the PCH system makes the data available to third parties such as researchers, drug companies, medical equipment manufacturers, and similar stakeholders in the health care field. The PCH system may provide the data freely, or in return for compensation.

One example of an analysis that may be made is to take a cohort of patients with the same medical condition and analyze the treatment provided by each patient's health care providers, and the outcomes achieved in each case. By comparing treatment choices and dosages against medical outcomes and side effects, the analysis is able to pinpoint an optimal treatment plan.

As another example, an analysis is made of different treatment plans with similar outcomes to pinpoint the most cost-effective treatment plan.

Optimal treatment plans thus identified are communicated to physicians via the PCH system. Similarly, patients are given access to the data, allowing them to make more informed decisions about their health care.

It is clear that the above examples are only a few examples of how access to a broad and deep database of health care information leads to improved efficiency in the delivery of health care and improved health care outcomes. Furthermore, access to the data may be in real time or near real-time, thereby improving the speed at which the state of the art advances.

X. Physician-Patient Needs Assessment and Unused Facilities

By analyzing the medical records of patients, the PCH system identifies geographical locations where specific treatments or types of physicians or specialists are needed. By comparing patient treatment needs, physician needs, or specialist needs to the PCH system records of medical facilities, the PCH system identifies over-use and under-use of facilities.

A physician searches the PCH system for regions where there is a need for his specialty and also searches for available facilities in the region with suitable equipment. Such facilities may be clinics, hospitals, laboratories, or other medical facilities. The physician may book through the PCH system a clinic or an operating room at such an available facility. The physician's clinic and availabilities are then made available on the PCH system for bookings by patients, as described in the “Appointment Options” section hereinabove.

A physician uses the PCH system to order equipment or supplies from a facility that has excess or unused inventory, to a facility that requires the specific equipment or supplies. Thus if a facility with available space is not currently equipped for a certain medical practice, the facility is made usable for the specific practice through the PCH system, by transferring items from another facility.

The PCH system also automatically identifies physicians' availabilities and automatically identifies suitable clinics and operating rooms with matching availabilities. The PCH system automatically notifies physicians of such availabilities and the physicians use the process described above to book a facility.

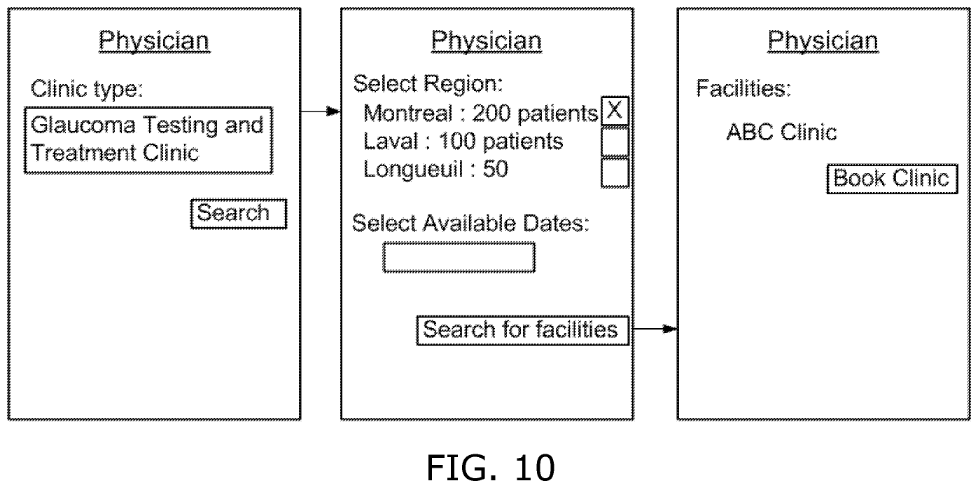

Reference is made to FIG. 10, which is a simplified series of screenshots showing how a physician books a clinic for a specific medical practice based on patient needs and available facilities, in accordance with an embodiment of the present invention. The physician searches for the type of clinic that he would like to offer. The PCH system returns a listing of patients waiting for an appointment for a specific medical issue treated at the clinic, based on geographical location. The physician selects the region or regions of interest and searches for available facilities for one or more dates when the physician is available. The physician is given a listing of available facilities and books a clinic on the PCH system. The clinic is then made available on the PCH system for patients to book an appointment.

It will be appreciated that by allowing a physician to book a clinic based on patient needs, location, and available facilities, that the wait time for patients is reduced.

XI. Real-Time and Historical Information

The PCH system analyzes real-time and historical data for the purpose of improving healthcare delivery efficiency. For example, the PCH system provides real-time analysis of emergency rooms in a geographical area. Such analysis includes analyzing patient traffic and determining current wait times at each hospital's emergency room. A centralized authority, such as a government where applicable, or a health care organization or hospital chain in other circumstances, then assesses where additional doctors are needed and allocates doctors based on need.

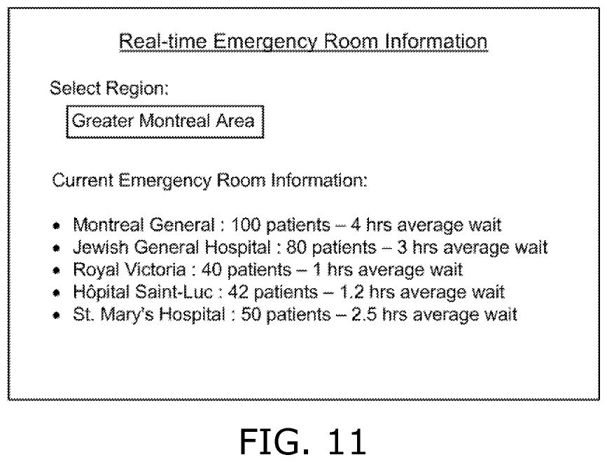

Reference is made to FIG. 11, which is a simplified screenshot showing real-time emergency room information for a given geographical location, in accordance with an embodiment of the present invention. For each hospital, the number of patients waiting to see an emergency room doctor and the average wait time are displayed.

The real-time analysis is used to notify patients of average wait times at different emergency rooms and recommends which emergency room to go to in order to have the shortest wait.

For a patient viewing emergency room wait times, the PCH system also provides a list of local doctors currently available. In some cases a person planning to go to an emergency room sees a nearby physician instead, if he is made aware that the possibility of seeing a nearby physician exists. This flexibility reduces wait times at hospital emergency rooms.

It is important to note that the flexibility of choosing an optimal emergency room or physician goes hand in hand with centralized patient profiles, as patients do not have to favor one emergency room or physician over another based on which provider has their records.

Historical analysis is also performed on medical facilities and facility staffing. The PCH system compares patient traffic and average waiting time at a facility with the staffing levels at the facility. The PCH system then makes staff scheduling recommendations that optimize patient wait times. Alternatively, the PCH system makes staff scheduling recommendations that optimize staff scheduling, including inter alia minimizing total staff work time while not exceeding a maximum permitted average patient wait time, or minimizing the number of shifts that staff members work while not exceeding a maximum permitted average patient wait time. In addition to optimizing use of facilities and staffing, such optimization also reduces administrative overhead due to reduced staffing and simplified staff schedules.

XII. Community Help

The PCH system is used to facilitate and organize volunteer efforts in a community.

An individual or a volunteer organization registers with the PCH system as being available for volunteer work, including providing parameters such as days and times available, geographical area limitations, and types of volunteer work, e.g. transportation assistance, home visits, hospital visits, medication delivery and patient advocacy.

Health care providers register volunteering needs with the system. For example, a hospital registers the need for hospital visits or home visits for a segment of the hospital's clientele.

The PCH system also analyzes data regarding congestion, patient needs, and resource availability, and suggest needs for volunteer efforts to providers automatically. For example, the PCH system may identify a large number of elderly or infirm patients in a particular area or visiting a particular clinic. As another example, the PCH system identifies a set of patients who have unfulfilled prescriptions, which indicates a need for assistance. The provider indicates to the PCH system if there really is such a need for assistance, and if so the opportunity is registered in the PCH system.

A volunteer browses available volunteering opportunities in the PCH system and signs up for ones he is interested in. Alternatively, the PCH system provides suggestions to volunteers based on the volunteer's parameters and the registered needs.

XIII. Emergency Responders

A patient uses the PCH system to request emergency medical assistance. The PCH system identifies a licensed emergency responder based on geographic location, availability, areas of expertise, or other considerations, and notifies the emergency responder. If the emergency responder chooses to assist, he is provided access to the patient's medical records based on permissions granted previously by the patient. Alternatively, the patient can, at the time of the emergency, grant access to emergency responders. If a patient is unable to access his profile during the emergency, access to the patient's records may be granted by the patient's representative, such as a family member, based on permissions established previously. As such the emergency responder has access to all relevant information about the patient even before arriving at the scene of the emergency.

If a patient calls for emergency assistance through traditional channels rather than through the PCH system, the responder, upon arriving on the scene, uses the PCH system to access any relevant medical information about the patient. As above, granting of access is based on permissions granted at the time or in advance by the patient or the patient's designated representative.

XIV. Disaster Management

In the event of a natural disaster such as a storm, earthquake or flood, the PCH system helps people get to safety.

The PCH system maintains a database of shelter locations for different types of emergencies. Shelter location information is maintained in advance, or in an actual emergency event relevant authorities enter shelter locations into the PCH system. For example, in a flood situation the relevant authorities designate a school on high ground as a gathering point for evacuees.

The PCH system sends an alert to users via their mobile devices indicating where to seek shelter or assistance. The information is sent based on geographical area in order to target specifically those users affected by the events. Similarly, information is sent indicating where medical treatment may be available and what types of medical facilities are available.

XV. Insurance

The system facilitates insurance processing.

When a patient undergoes a covered medical treatment or procedure, the provider creates a record in the patient's profile indicating that the treatment or procedure was carried out. The PCH system provides the treatment or procedure information to the relevant insurance companies directly.

A patient may specify that medical information be provided automatically to the relevant insurance companies, or that medical information be provided only upon the patient's directive.

A patient may maintain financial information, such as bank account information, in his profile.

If a patient has an out-of-pocket covered expense for a medical treatment the PCH system may, at the same time the PCH system sends the relevant medical information to the insurance company, also send to the insurance company a request for patient reimbursement to the previously provided bank account.

Alternatively, a patient may require a treatment that requires pre-approval by the insurance company. In such case the PCH system sends information about the requested treatment to the relevant insurance company, along with any required patient health and medical records, and the insurance company notifies the PCH system if the request for coverage is approved. If the request is approved, the PCH system sends the approval both to the patient and to a physician, clinic, or hospital that will be providing the treatment.

In the foregoing specification, the invention has been described with reference to specific exemplary embodiments thereof. It will, however, be evident that various modifications and changes may be made to the specific exemplary embodiments without departing from the broader spirit and scope of the invention. Accordingly, the specification and drawings are to be regarded in an illustrative rather than a restrictive sense.

1. A system for patient-centric health care, comprising:
a plurality of client computing devices configured to store medical records, and to provide a plurality of interactive patient medical management systems (MMSs) for each of a plurality of patients, wherein access to each patient MMS by entities other than the respective patient is controlled by the respective patient; and
a centralized host server configured to store medical records and to provide communication interfaces with a plurality of interactive MMSs for each of a plurality of patients, hospitals, clinics, medical test centers, physicians, pharmacies, insurance companies and patients,
wherein said client computers and said centralized host server comprise processors and instructions that enable each patient to perform the following processes, via the patient's MMS: (a) share patient medical records with hospitals, clinics, medical test centers, physicians, pharmacies, insurance companies or emergency responders, (b) schedule appointment with a physician, (c) schedule an appointment with a specialist based on a referral from a physician, (d) schedule a medical test based on a referral from a physician, (e) transmit a prescription prescribed by a physician to a pharmacy for fulfillment, and (f) generate an insurance claim.

2. The system of claim 1 wherein said instructions enable each physician to perform the following processes, via the physician's MMS: (a) access records stored in a patient MMS, (b) transmit to the patient MMS a referral of the patient to a specialist, (c) transmit to the patient MMS a referral of the patient for a medical test, (d) transmit to the patient MMS an interpretation of results of a medical test, (e) transmit to the patient MMS a prescription for a patient, (f) transmit to the patient MMS a physician's report, and (g) transmit to the patient MMS a physician's invoice.

3. The system of claim 1 wherein said instructions enable each hospital to perform the following processes, via the hospital's MMS: (a) access records stored in a patient MMS, (b) reserve a facility for a surgeon selected by the patient, (c) reserve equipment for a surgeon selected by the patient, (d) reserve a hospital room for the patient, (e) transmit to the patient MMS a hospital report, and (f) transmit to the patient MMS a hospital invoice.

4. The system of claim 1 wherein said instructions enable each pharmacy to perform the following processes, via the pharmacy's MMS: (a) access records stored in a patient MMS, (b) receive a prescription from the patient MMS, and (c) coordinate delivery of the fulfilled prescription to the patient.

5. The system of claim 1 wherein said instructions enable each test center to perform the following processes, via the test center's MMS: (a) access records stored in a patient MMS, (b) receive a referral for the patient, (c) schedule an appointment in conjunction with the patient MMS, and (d) transmit test results to the patient MMS.

6. The system of claim 1 wherein said instructions enable each insurance company to perform the following processes, via the insurance company's MMS: (a) receive from a patient's MMS a record for a medical treatment covered by the patient's insurance policy, (b) generate an insurance claim, (c) generate a payment instruction in accordance with the insurance claim, (d) receive a treatment request from the patient's MMS, and (e) approve or reject the treatment request.

7. The system of claim 1 wherein said centralized host server performs data analysis on patient medical records to infer optimal treatment plans and cost-effective treatment plans, and provides the results of the analyses to patient MMSs, hospital MMSs, clinic MMSs, test center MMSs, physician MMSs and insurance company MMSs.

8. The system of claim 1 wherein said centralized host server performs data analysis to identify over-utilized and under-utilized facilities, and provides the results of the analysis to patient MMSs, hospital MMSs, clinic MMSs, test center MMSs, physician MMSs and insurance company MMSs.

9. The system of claim 1 wherein said centralized host server performs data analysis to determine average wait times for emergency rooms, and provides the results of the analysis to patient MMSs, hospital MMSs, clinic MMSs, test center MMSs, physician MMSs and insurance company MMSs.

10. The system of claim 1 wherein said centralized host server performs data analysis to determine places with needs for volunteer effort, and provides the results of the analysis to potential volunteers.

11. A system for patient-centric health care that provides each of a plurality of patients, via a medical management systems (MMS) from a corresponding plurality of MMSs, access to the MMS by entities other than the patient being controlled by the patient, with access to his medical records, comprising physician reports, hospital reports, test results, treatments, medications, prescriptions and insurance claims, and enables each patient, via the MMS controlled by the patient, to share access to at least a portion of his medical records with hospitals, clinics, medical test centers, physicians, pharmacies, insurance companies and emergency responders.

12. The system of claim 11 further enabling each patient, via the MMS controlled by the patient, to grant permission to hospitals, clinics, medical test centers, physicians, pharmacies, insurance companies and emergency responders, to edit at least a portion of his medical records and to store additional medical records.

13. The system of claim 11 further enabling each patient, via the MMS controlled by the patient, to selectively grant permission to hospitals, clinics, medical test centers, physicians, pharmacies, insurance companies and emergency responders to share designated portions of his medical records with other hospitals, clinics, medical test centers, physicians, pharmacies, insurance companies and emergency responders.
